(19) United States
(12) Patent Application Publication
(10) Pub. No.: US 2021/0244092 A1
(43) Pub. Date: Aug. 12, 2021

(54) VAPORIZING SYSTEM AND DISPOSABLE CAPSULES FOR USE THEREWITH

(71) Applicant: CTH LTD., Ness Ziona (IL)

(72) Inventors: Isaac Weigensberg, Holon (IL); Gregory Kunin, Rehovot (IL); Yuval Malka, Dvira (IL); Arie Kalo, Ness Ziona (IL)

(21) Appl. No.: 17/052,697

(22) PCT Filed: Jun. 4, 2020
(86) PCT No.: PCT/IL2020/050625

§ 371 Date: Nov. 3, 2020

(30) Foreign Application Priority Data
Jun. 5, 2019 (IL) 267118

(51) Int. Cl. A24F 40/42; A24F 40/46; A24F 40/20; A24F 40/53; A24F 40/51; A24F 7/02; H05B 3/10; H05B 1/02; G05D 23/24; G06K 7/14

(52) U.S. Cl. CPC A24F 40/42; A24F 40/46; A24F 40/20; A24F 40/53; G06K 7/1413; A24F 7/02; H05B 3/10; H05B 1/0252; G05D 23/2401; A24F 40/51

(57) ABSTRACT

The present invention provides disposable capsules and electronic inhalers designed to be used therewith, and to methods for consuming cannabis in a clean, simple and efficient way, which is also cost effective.

### FIELD OF THE INVENTION

The present invention relates to devices and systems for delivering at least one active substance by using disposable capsules holding an herbal mixture containing the active substance.

### BACKGROUND

Cannabis commonly used both for recreational and medical purposes for thousands of years. However, in the early 1900s, US passed regulatory laws regarding cannabis growing and usage. The Controlled Substances Act of 1970 classified cannabis along with heroin and LSD as a Schedule I drug, which revoked acceptance of cannabis for medical use. This course of things continued until recognition of the benefits of medical marijuana began at the late 20th century.

The medical benefits attributed to marijuana are broad and include, e.g., treatment of innumerable conditions such as pain, chronic inflammation, anxiety, depression, Crohn's disease, Parkinson's disease, etc. These health benefits are the basis for the marijuana legalization movement for both medical and recreational.

The two most common techniques for consuming cannabis are inhalation (i.e., via the lungs) and direct consumption (i.e., via the stomach). Inhalation is considered to be a more effective e method since it has an effects within a few seconds of consumption even at low dosages. On the other hand, direct consumption of cannabis takes a few hours to generate the same or similar effects, which may lead to overdosing. An alternative usage of cannabis is the use of cannabis oil by ingestion and/or transdermal application.

The common method of inhalation is smoking, namely placing dried cannabis-based dried herb in a pipe or rolled in a cigarette paper, igniting it, and inhaling the smoke. However, this method involves the consumption of other combustion residues, such as smoke, carbon monoxide and various carcinogens, e.g., due to the burning of the paper. Notably, since most cannabis smokers do not use filter, they are more susceptible to increased tar deposits in the lungs, which contributes to respiratory damage.

When a user inhales smoke, e.g. via a cigarette, the smoke and tar herein may cause various respiratory problems and long term damage. When smoke comes in contact with the airway passage and lungs it might cause visible and microscopic injuries. As a result, smokers develop problems such as cough, increased phlegm production, wheezing, bronchitis, acute chest illnesses, and risk of lung infections.

An alternative, more “clean” smoking is to inhale vapors. Vaporization is one method of consuming cannabis without the damaging smoke and which limits the toxins entering the airways. In this technique, the herb-based substance is heated to a temperature of about 180-190° C. in which vapors are formed, which is below the combustion temperature of cannabis, i.e., below about 230° C., where noxious smoke and associated toxins are produced. Since vaporization allows the user to receive doses of cannabinoids while reducing the intake of carcinogenic smoke, it is considered to be one of the more preferred methods of cannabis administration.

A recent development is the electronic cigarette for nicotine consumption. It was also adapted for consuming cannabis—in an electronic cannabis cigarette adjusted to heat cannabis oil at a specific temperature. In comparison to traditional cannabis smoking, such electronic cannabis cigarettes are considered safer and healthier since the inhaled vapors do not include tar and carcinogens. Notably, cannabis oil is more viscous than nicotine liquid, which might clog and prevent cannabis oil flow. Hence, conventional electronic cigarettes, vape-pens and other oil-vaporizer devices require continuous upkeep, limit portability, and lack discreetness while being relatively expensive.

In recent years many developments in the field of electronic cigarettes can be seen. For instance, US 2012/0273589, US 2014/0209110, EP 2840913, WO 2013/020220, U.S. Pat. Nos. 9,282,772 and 8,528,569 describe various configurations of electronic cigarettes and elements thereof.

U.S. Pat. No. 9,814,262 describes an electric cigarette with thermal management system for monitoring and controlling the thermal properties of the electric cigarette for optimal conditions for atomization and smoke generation.

WO 2008/026155, US 2017/0238617, US 20160192708 and “Juul plans . . . ”, 2018 (D6) disclose inhalers and pods that hold a liquid material, which is completely different from a solid herb-based filing. The difference between solid and liquid filing means that the overall structure and mechanism-of-action is different and one cannot simply take a liquid-filing pod and replace the liquid with a dry substance with any expectation that it will still work or that it will work with the same efficiency.

WO 2016/001925 provides an open pod (dose cartridge) for an electric inhaler, the pod comprises a flat pallet holding drug-substance, the pallet is made of airflow permeable material that allows air to pass therethrough and over/through the drug-substance. Due to the flat shape of the pod, it needs a support (supporting element), which further includes a heater, which means that the heater is external, and is in fact located outside the pod. Moreover, such a configuration limits the heater to be U-shaped or rectangular-shaped (i.e. along the edges of the supporting element). Due to the structure and location of the heater, the heating affects mainly the material that is in direct contact with the heater, while remote or inner material is insufficiently heated, which reduces the efficiency of the heater, may result with reduced amount of released active substance and/or in burning of the dried content. Moreover, the structure of the electric inhaler that uses such an open-pod that allows airflow to pass through the surface of the pod, means that aerosolization is not isolated from the non-reusable part/component, which means that the inhaler will require cleaning after each use to avoid contamination risk.

### SUMMARY

In one embodiment, the present invention provides a pod/capsule 200 comprising a capsule part filled with an herb-based filling with an herb-based active substance, suitable for inhaling said active substance using an electric inhaler 100. In specific embodiments, the capsule part of the pod 200 comprises (a) an envelope 202; and (b) a heater 201 disposed within said envelope 202.

In another embodiment, the present invention provides a sealed/enclosed pod/capsule 200 comprising: (i) an envelope 202 filled with a solid herb-based filling with an herb-based active substance, suitable for inhaling said active substance using an electric inhaler 100; and (ii) a heater 201 disposed within said envelope 202, wherein (i) said envelope 202 includes an airflow inlet and an airflow outlet designed to direct and allow passage of air via said envelope 202; and (ii) said heater 201 is designed to heat all said solid herb-based filling at once.

In yet another embodiments, the present invention provides a pod/capsule 200 suitable for use with an electric inhaler 100, said pod 200 comprising: an envelope 202 and a heater 201, wherein: (i) said envelope 202 comprises a thermally isolated cavity, said cavity contains a solid herb-based filling with an herb-based active substance; (ii) said envelope 202 comprises an airflow inlet and an airflow outlet designed to direct and allow passage of air via said cavity; and (iii) said heater 201 is disposed within said cavity, and is designed to heat the entire cavity and said solid herb-based filling.

In yet another embodiment, the present invention provides an electric inhaler 100 for inhaling vapors of an herb-based active substance encapsulated/stored within a pod/capsule 200 as defined above holding an herb-based filling with said active substance, wherein said electric inhaler 100 comprises: (a) a hollow body having an airflow inlet and outlet 104 connected via an airflow path; (b) a pod/capsule socket/receptacle 105 within said body adapted to receive and hold said pod/capsule 200; (c) a mouthpiece located at the airflow outlet 104; and (d) an electrical power control switch, wherein the inhaler 100 is designed to receive and hold a pod/capsule 200 as defined above, and activate the heater 201 therewithin using an electric power source 101 to thereby release the active substance from the herb-based filling

In another embodiment, the present invention provides an electric system for smoking/consuming/inhaling an herb-based active substance, said system comprising: (a) a pod/capsule 200 as defined above; (b) an electric inhaler 100 as defined above; and (c) a power source 101 (optionally rechargeable), optionally integrated within said electric inhaler 100.

In another embodiment, the present invention provides a method for consuming an herb-based active substance, said method comprising the steps of: (i) placing a pod/capsule 200 as defined above within a pod/capsule socket/receptacle 105 in an inhaler 100 as defined above; (ii) activating the heater 201 within said pod/capsule 200 to thereby release said herb-based active substance from the herb-based filling; and (iii) inhaling the released active substance by inhaling via the mouthpiece of the inhaler 100.

### BRIEF DESCRIPTION OF THE DRAWINGS

FIGS. 1A-1B are 3-dimensional illustrations of an electronic inhaler according to some embodiments of the invention. FIG. 1A is a 3-D image of the inhaler, FIG. 1B is a 3-D image of the inhaler with a pod/capsule positioned at the opening of the socket/receptacle of the inhaler; and FIG. 1C is a cross-sectional side-view thereof with a pod/capsule therein.

FIGS. 2A-2C illustrate yet another possible configuration of an electronic inhaler according to some embodiments of the invention: FIG. 2A illustrates the inhaler and pod before assembly; FIG. 2B illustrates the inhaler and pod after assembly; and FIG. 2C illustrates the inner components of the inhaler and pod after assembly.

FIGS. 3A-3B illustrate an electronic inhaler according to some embodiments of the invention with a removable disposable mouthpiece: FIG. 3A illustrates an assembled inhaler; and FIG. 3B illustrates the inhaler with the removable mouthpiece extracted outwardly.

FIGS. 4A-4B illustrate the components of an exemplary pod/capsule according to some embodiments of the invention: FIG. 4A is a cross-sectional side-view of an exemplary pod with a heater around a solid filling; and FIG. 4B is a bottom-view thereof.

FIGS. 5A-5C are exploded views illustrating two possible configurations of a pod/capsule according to some embodiments of the invention.

FIGS. 6A-6B illustrate one possibility of making a single-use disposable pod that cannot be reassembled after dissembling.

FIGS. 7A-7E illustrate the different components of a pod/capsule according to some embodiments of the invention from different viewing angles.

### DETAILED DESCRIPTION

Cannabis usage is a growing field for both recreational and medical use. However, common consumption techniques involve health risks and/or require cumbersome preparations before use, e.g. using a cannabis cigarette. Other techniques, such as electronic cannabis cigarettes are expensive and require purchasing of expensive dedicated cassettes with fillings, usually cannabis oil. However, known electronic cigarettes have various disadvantages such as any of the following: slow degradation/failure of the heating element due to prolong use and accumulation of residues thereon; the need to extract the oils from the herb, which involves more complicated processing and often leads to changes in flavor and constituents of the final product.

Contrary to the known electronic cigarettes, the electronic inhaler according to the invention overcomes all the disadvantages of the prior art by using disposable pod/capsule with an integral heater and pre-packed with herb-based substance. When such a pod/capsule is placed within the inhaler, the integral heater heats the pre-packed herb-based substance according to a predefined program (according to desire).

Accordingly, in a first aspect, the present invention provides a pod/capsule 200 comprising a capsule part filled with a solid herb-based filling with an herb-based active substance, suitable for inhaling said active substance using an electric inhaler 100.

Known pods, e.g. as disclosed in WO 2008/026155, US 2017/0238617 and US 2016/0192708, disclose inhalers and pods that hold a liquid substance, which are inadequate for use with a solid filing.

In certain embodiments, the present invention provides a sealed/enclosed pod/capsule 200 comprising: (i) an envelope 202 filled with a solid herb-based filling with an herb-based active substance, suitable for inhaling said active substance using an electric inhaler 100; and (ii) a heater 201 disposed within said envelope 202, wherein (i) said envelope 202 includes an airflow inlet and an airflow outlet designed to direct and allow passage of air via said envelope 202; and (ii) said heater 201 is designed to heat all said solid herb-based filling uniformly and at once.

In certain embodiments, the pod of the present invention is a sealed/enclosed pod. Open pods, such as disclosed in WO 2016/001925, require a separate enclosure to maintain heat and any generated heat affects mainly the material that is in direct contact with (or in close proximity to) the heater, while remote or inner material is insufficiently heated, which reduces the efficiency of the heater, may result with unburned residues, reduced amount of released active substance and/or in burning of the dried content (at the contact point with the heater). However, the instant closed/sealed pod enables to maintain all the heat generated by the heater inside the pod, which allows for an even and faster heating of all solid content. Moreover, passing aerosols and burned material and vapors through a permanent, non-disposable part/component of the inhaler forces repetitive cleaning thereof after each use to avoid risk of contamination.

Accordingly, in certain embodiments, the present invention provides a pod/capsule 200 suitable for use with an electric inhaler 100, said pod 200 comprising: an envelope 202 and a heater 201, wherein: (i) said envelope 202 comprises a thermally isolated cavity, said cavity contains a solid herb-based filling with an herb-based active substance; (ii) said envelope 202 comprises an airflow inlet and an airflow outlet designed to direct and allow passage of air via said cavity; and (iii) said heater 201 is disposed within said cavity, and is designed to heat the entire cavity and said solid herb-based filling.

In certain embodiments of the pod/capsule 200 of the invention, the thermally isolated cavity enables to maintain the pod's outer surface at a temperature that is safe for a person to touch even during activation of the heater 201, such as at ambient temperature or a temperature that is below 60° C., e.g. about 55° C., 50° C., 45° C., 40° C. or less. This enables the user to touch and discard the used pod 200 immediately after use without the risk of getting a burn. Moreover, the fact that the pod's external/outer surface is maintained relatively low, compared to the temperature generated inside the pod by the heater 201 to release the active substance from the solid herb-based filing, means that an inhaler using such a pod 200 is in fact fire-safe, since there is no risk of overheating the environment or the surface onto which the inhaler is placed.

Such cavity heat-isolation can be reached by any suitable way, such as using heat-isolation material for the cavity walls, adding a heat-isolation layer to the cavity's walls, and/or fabricating air pockets within the cavity's walls.

This cavity heat-isolation enables touching the surface of the pod 200 during use without risking being burned. Such heat-isolation also improves the uniform heating capabilities of the content of the pod (the filling) and reduces heat dissipation out of the pod, which in turn aids in better power efficiency.

In certain embodiments of the pod/capsule 200 of the invention, the solid herb-based filling is held essentially entirely by said heater 201.

In certain embodiments of the pod/capsule 200 of the invention, the solid herb-based filling is not in direct contact with the heater 201. This is enabled due to the presence of the sealed envelope that enables heating the air within the pod, which enables even heating within the pod and eliminates the necessity of direct contact between the heater and the solid herb-based filing. Moreover, this also allows setting a higher temperature of the heater to facilitate faster evaporating of the active substance from the solid herb-based filling. Furthermore, setting the heater to a higher temperature is advantageous as it allows faster heating of the air inside the pod and avoiding overheating only a part of the filling. In alternative embodiments of the pod/capsule 200 of the invention, the solid herb-based filling is in direct contact with the heater 201.

In specific embodiments of the pod 200 according to any of the embodiments above, the heater 201 is shaped to enable efficient heat conduction to all areas of the filling, such as cylindrical-shaped; spring-like-shaped; serpentine-shaped; rod- or multiple rods-shaped; or cubical-shaped.

In certain embodiments of the pod/capsule 200 of the invention, air that flows from the envelope's inlet to its outlet does not pass through the solid herb-based filling. This is enabled by creating air passageway that directs air entering the pod from the air inlet to the air outlet, wherein vapors generated within the pod's cavity (due to heating of the filling) enter this air passageway via dedicated passages due to, e.g., vacuum forces of the passing air and/or increased pressure of the generated vapors during the heating of the filling. This is also enabled by the presence of the sealed envelope, and allows using tighter packed solid filling, which simplifies the process of filling the pod with the dried herb.

The terms “pod” and “capsule” as used herein interchangeably refer to any type of a sealed container that can hold herb-based active substance(s). The pod may be formed from a single piece of material, such as plastic, polymer or metal. The design of the capsule is tailored to fit the inner dimensions of an opening within a dedicated inhaler 100, such that it is held in place until pulled out. It should be noted that the pod can be in any shape and size, such as a cube, a box, a pyramid, a cylinder, etc. The size of the pod can be determined according to the amount of filling intended for use. In specific embodiments, the pod 200 is cubic.

The term “consuming” as used herein refer to the consumption of the active substance(s) released from the herb-based filling within the pod 200 of the invention. The consumption may be by “smoking”, e.g., burned/combusted herb-based filling or by inhaling vapors of heated herb-based filling without burning thereof.

The term “solid” as used herein with reference to the herb-based filling relates to the state of matter of the filling, and which is not required to change shape during use and/or before evaporation. As such, the term “solid” includes solid as well as semi-solid such as wax and jelly.

In certain embodiments, the capsule 200 is made of an opaque and airtight material to prevent penetration of humidity, light and/or oxygen, and assures maximal shelf life expectancy to the active substance(s) within. Alternatively, the capsule 200 is not made of an opaque and/or airtight material but is sealed within an opaque and airtight package to obtain these advantages.

In certain embodiments of the pod 200 according to the invention, the herb-based filling is selected from: dried herb(s), preferably unprocessed and without additives; and wax composed of mainly extracts or derivatives from such herb(s). The herb can be either a single herb or a mixture of different herbs in order to obtain, e.g., unique taste, aroma and/or combination of active substances. It should be noted that any suitable way for preparing the herb-based filling is within the scope of the invention, including, but not limited to, drying (e.g. dry-freeze or heat-drying) the herb, preparing extract from different components of the herb, boiling, etc.

In specific embodiments, the herb-based filling is dried herb(s) packaged tight in the capsule part to improve heat conduction. As such, it is to be noted that the filling is steady still and does not move across the heater 201, while the heat from the heater 201 is efficiently conducted to all pans/areas of the filling to achieve optimal and maximal release of the active substance from all the filling.

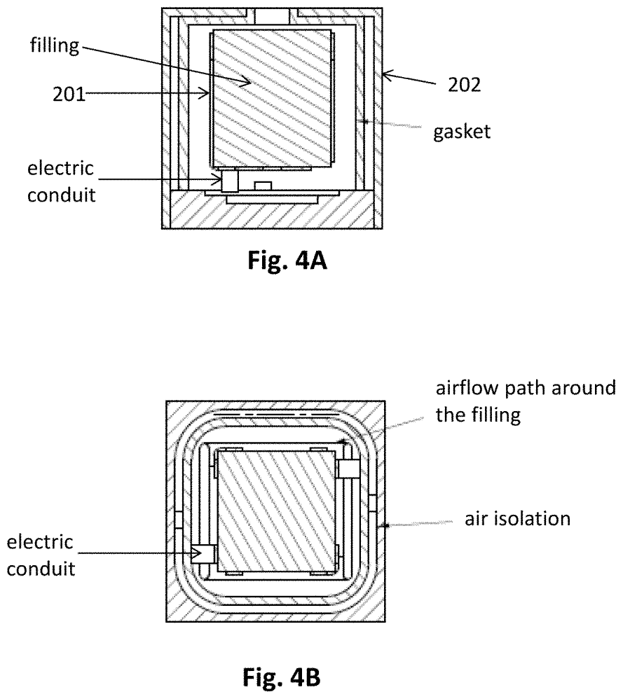

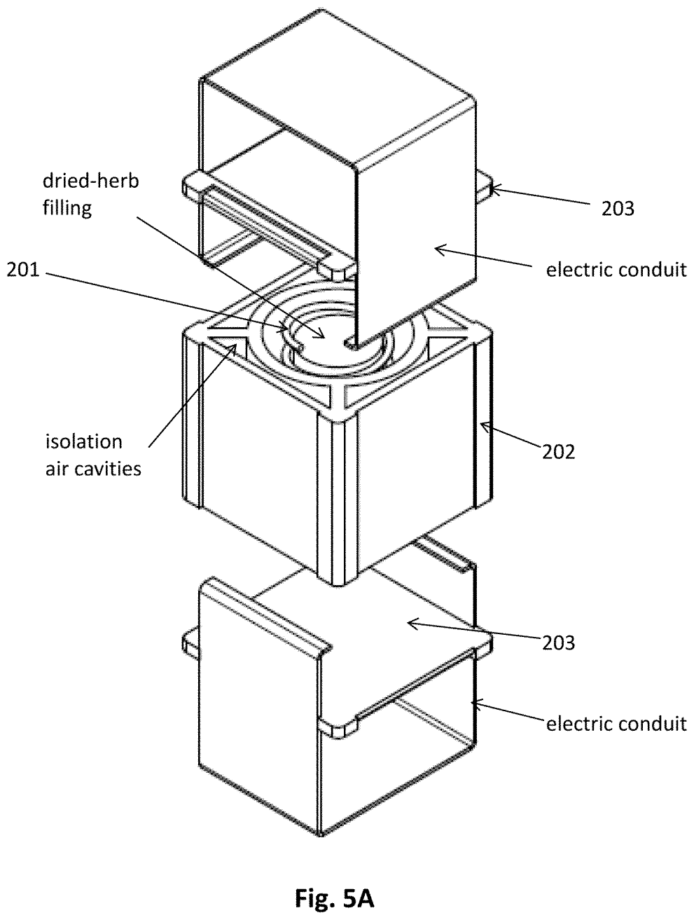

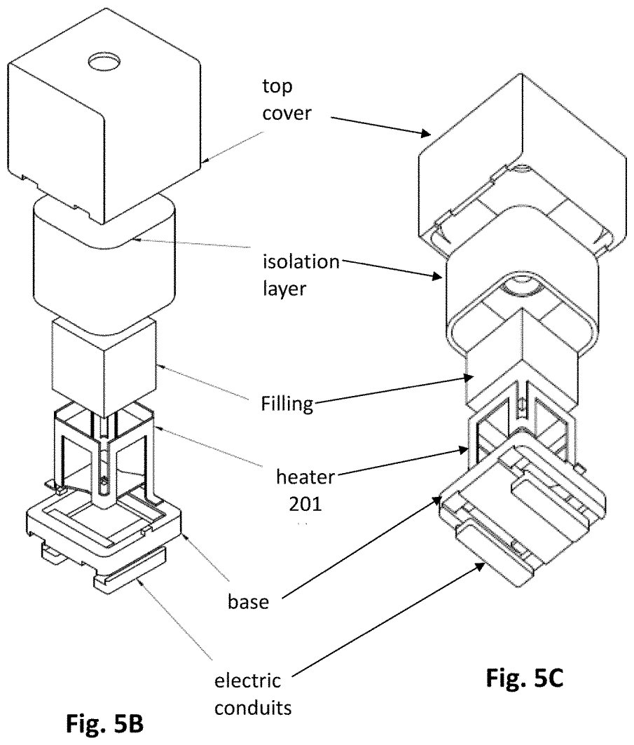

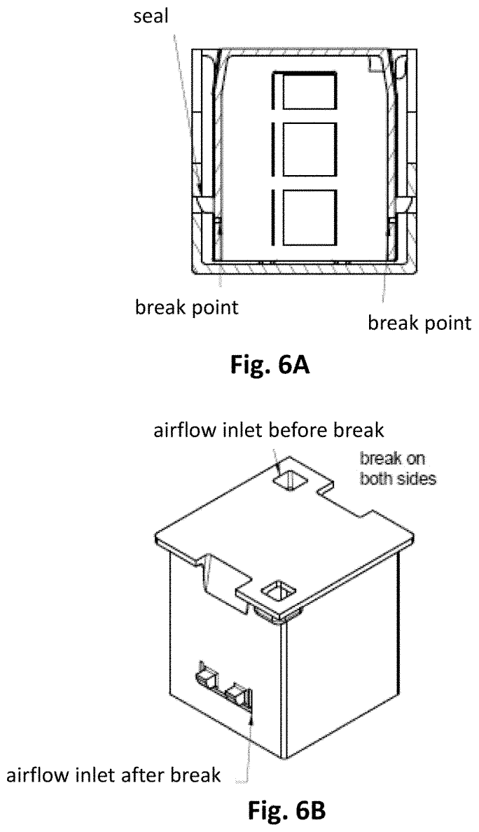

In certain embodiments of the pod 200 according to any of the embodiments above, the shape of the heater 201 is cylindrical; spring-like (see e.g. FIG. 5A); serpentine; rod-shaped; multiple rods; cubical-shaped, (see e.g. FIGS. 4 & 5B), U-shaped (see e.g. FIG. 6), or any other shape or construct that enables efficient heat conduction to all areas of the filling. The heater may constitute a single element or a combination of individual heating elements linked to work together (e.g. several rods or heating stripes). In certain embodiments, the heater 201 is made of a coiled wire; a woven wire; stainless steel mesh; sheet metal (punched or etched). The material from which the heater is made can be determined according to the type and shape of the filling and according to the desired final heat.

In certain embodiments, there is direct contact between the heater 201 and the herb-based filling. In alternative embodiments, there is no direct contact between the heater 201 and the herb-based filling, e.g. there is an electrically conductive or non-conductive surface between the heater 201 and the filling. Non-limiting examples of such non-conductive surfaces are ceramic, glass or anodized metal.

In certain embodiments, the filling is within the heater 201. In specific embodiments thereof, the heater is in direct contact with the filling. In alternative specific embodiments, the heater is not in direct contact with the filling.

In certain embodiments, the heater 201 is within the filling. In specific embodiments thereof, the heater is in direct contact with the filling. In alternative specific embodiments, the heater is not in direct contact with the filling.

In specific embodiments, the heater 201 goes through the filling, i.e. wrapped around by the filling. In yet other alternative embodiments, the filling is surrounded by the heater 201. In further specific embodiments, the filling is both inside and outside the heater 201 (e.g. inside and around a spiral heater). In further specific embodiments, the heater 201 is both inside and outside the filling (e.g. spiral-shaped heater that passes within the filling together with a cubic-shaped heater wrapping the filling).

In certain embodiments, the capsule part comprises external electrical contact(s) designed to connect an external power source 101 (e.g. of an inhaler) to the heater 201 for activation thereof.

In certain embodiments, the herb-based filling is prepared separately and is incorporated into the pod 200 during assembly thereof. In alternative embodiments, the herb-based filling is fabricated directly into the assembled pod 200, e.g. by pressing, injecting, molding, etc.

In certain embodiments, the herb-based filling comprises only components from the herb, i.e., leaves, stems, flowers, etc., either from a single plant species or several. In alternative embodiments, the herb-based filling further comprises additives, such as for improving heat conductivity, aerosol generation, smell, taste, and/or anti-bacterial properties (e.g. preservatives).

In certain embodiments, the pod/capsule 200 according to any one of the embodiments above further comprises an indicator, such as barcode or other type of memory (e.g. eeprom —electricity erasable programmable read only memory), for identification purposes. i.e. to identify at last one of: the pod/capsule type; the nature, type and amount of the herb-based filling, usage temperature profile, heater resistance at room temperature, production/expiration date, batch number, and tractability information.

In certain embodiments of the pod/capsule 200 according to any one of the embodiments, the capsule part comprises: (a) an envelope 202; and (b) a heater 201 disposed within said envelope 202. In specific embodiments, the capsule part comprises an insulation layer to prevent heating of the surroundings of the pod/capsule 200 and/or to prevent burning of a user's hand when removing the pod/capsule 200 from an inhaler, when/after the heater 201 is activated. In certain embodiments, the heater 201 is isolated from the capsule part, e.g. there is an insulation air pocket between the heater 201 and the walls of the capsule part. In alternative embodiments, the surrounding walls of the pod comprise isolation elements, such as air pockets or are made of heat-isolated material.

It should be understood that the pod 200 according to the invention may be made/fabricated from any suitable material and in any suitable way/technique, such as blow molding, press molding, 3D printing, etc. Accordingly, the incorporation of any isolation elements may be done during manufacture of the pod 200, or added thereafter, e.g. by coating the walls of the envelope 202.

In certain embodiments, the pod 200 is manufactured from rigid plastic material(s). In alternative embodiments, the pod 200 is made from brittle material that breaks if someone tries to open the pod (e.g. for refilling and reuse).

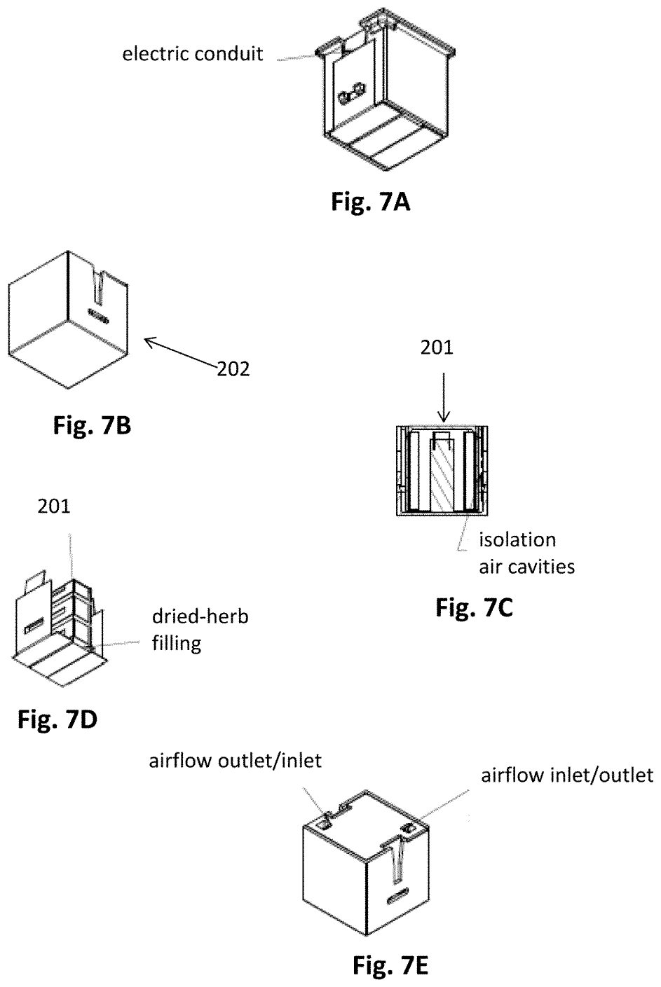

In certain embodiments, the pod 200 of the invention is disposable and is designed for a single use by preventing the possibility of reassembling after dissembling it. This is possible, e.g., by generating air pocket insulations that are breached when force opening the pod 200, and/or by constructing an airflow-path from the inlet to the outlet in the pod, which is destroyed when the pod 200 is forced opened (illustrated in FIG. 6): before breaking/opening the pod, the air flows from one side of the filling to the other; however, ater breaking/opening the pod, the seal within the pod breaks causing air to flow directly from the inlet to the outlet without passing through the filling. FIG. 6A is a cross-sectional side-view of an exemplary disposable pod according to the invention showing the presence of a seal at designated openings. When the pod is intact, the seal is intact thereby preventing passage of air flow via these openings and only enables air to flow from a designated inlet(s) via the filling (to collect fumes/vapors) and out through the outlet(s) to the user during inhalation. However, after force opening the pod, the seal breaks thereby creating free passage for air to flow through and out of such openings (referred to as “airflow inlet after breaking” in FIG. 6B). When air passes through these openings, it does not go via the filling, thereby preventing delivery of vapors/fumes to the user. FIG. 7 illustrates another possibility, in which once the cover of a pod is removed, all pars fall apart and cannot be re-joined (e.g. when the cover is made of pressed paper without a binder).

In specific embodiments of the pod 200 of the invention, the envelope 202 is heat-isolated, i.e. in addition to the heat-isolation capabilities of the cavity. In further specific embodiments, the envelope 202 comprises air cavities acting as heat-insulates. In yet further specific embodiments, i.e., when the envelope 202 comprises air cavities, the pod 200 further comprises sealers 203 designed to maintain the air within the air cavities for isolation purposes. Such sealers may be placed at any time point during manufacture of the pod 200, e.g. before or after placing the filling therein. In specific embodiments, the This heat-isolation enables touching the surface of the pod 200 during use without risking being burned. Such heat-isolation also improves the uniform heating capabilities of the content of the pod (the solid herb-based filling) and reduces heat dissipation out of the pod, which in turn aids in better power efficiency. Better power efficiency means that a smaller battery and device are required and/or longer use per charge.

In certain embodiments, the pod 200 of any of the above embodiments, further comprises sealers 203 designed to direct and allow passage of air only via the envelope 202 and/or the filling. In a specific embodiment, the sealers used to maintain air within the air cavities within the envelope 202 are the same sealers used to direct and allow passage of air only via the envelope 202 and/or the filling.

In certain embodiments of the pod/capsule 200 of the invention, the envelope 202 and/or cavity thereof is made-of, or comprises, a heat-isolation layer. In specific embodiments, the heat-isolation layer is made-of a plant/cellulose-based substance.

A pod 200 according to the invention comprises a heater 201 that is designed to generate the required heat for releasing active substances from the herb-based filling. As such, in specific embodiments, the heater 201 is designed to provide uniform heating of the herb-based filling. This uniform heating can be achieved by evenly spacing heater 201 components across the filling, or by using heating to generate higher temperature at areas more remotely from the heater surface, or by using heat-isolation envelope, or any combination thereof. Such even heating is beneficiary for immediate and complete release of the active substance from the filling without exposing “used” areas (from which the active substance has already been withdrawn) of the filling to unnecessary heat which might result in release of undesired substances.

Since the active substance(s) released from the solid herb-based filling, exits the pod 200, and flows through an airway passage within a suitable inhaler, the pod 200 needs to allow passage of air in and out. Accordingly, in certain embodiments of the pod 200 according to any of the embodiments above, (a) the heater 201 is permeable to vapor flow, i.e. the airflow passes through a narrow region around/within the heater 201, optionally via said filling; and (b) the herb-based filling is positioned around and/or within the heater 201. In specific embodiments, the herb-based filling is held by said heater 201.

In certain embodiments, the pod 200 is made of or comprises at least one wall/region that is permeable to air, to thereby allow for passage of air and of vapors/fumes of the active substance(s) released from the filling after activation of the heater 201. Said permeable wall/region of the pod may be impermeable during storage and transfer into permeability during usage, e.g. due to heat from the heater 201. Alternatively, it may be permeable, in which case the pod 200 may be stored within a sealed package creating an isolated environment for preservation purposes during storage. In a further specific embodiment, several pods 200 are stored separately from one another in a magazine or tablet-packet enabling discharging individual pods 200 when needed.

In certain embodiments, the pod/capsule 200 of the invention further comprises a one-way valve that allows passage of air through the pod only on inhalation. This prevents waste of released active agent(s) due to their discharge to the environment, as happens in open devices where the fumes/vapors are released to the air when not inhaled by the user. This further allows for a better heat retention within the pod and subsequently improves power efficiency.

Reaching the right temperature within the pod 200 is important to achieve complete extraction of the active substance from the filling. Insufficient heat may result in low amount of released active substance(s), and excessive heat may burn the filling and result in undesired burn-materials. Accordingly, in certain embodiments, the pod 200 according to any one of the embodiments above further comprises at least one element made of a material that enables temperature measurement inside the capsule 200. In specific embodiments, such an element is the heater itself having measurable temperature coefficient of resistance. In alternative embodiments, the element is an internal additional element residing within the pod 200 which enables electrical temperature measurements such as a thermocouple or resistance thermometer (RTD).

Some of the main advantages of the consumable pod 200 of the invention pre-filled with solid herb-based filling are: (1) no need to clean, hassle-free-all the leftovers stay within the pod 200 after use; (2) no waste of dry herbs by the filling process; (3) heating efficiency, no barriers between the heating element and the filling and less losses; (4) heating consistency; (5) freshness—the amount of filling inside each pod 200 is enough for one session, so a fresh pod 200 can be used in each session; (6) pod recognition that enables adjusted operation method per pod type; (7) no contamination between sessions; and (8) consistent experiences for different pods.

In certain embodiments of the pod 200 of the invention, the airflow goes through a narrow region around the heater 201 and/or the filling.

In specific embodiments the present invention provides a pod/capsule 200 comprising a capsule part comprising: (a) an envelope 202; and (b) a heater 201 designed for uniform heating of the herb-based filling and disposed within said envelope 202, the envelope 202 being filled with a dried herb(s) tight-packaged filling with an herb-based active substance, suitable for inhaling said active substance using an electric inhaler 100, wherein the filling optionally comprises additives for heat conductivity, aerosol generation and/or anti-bacterial properties. In further specific embodiments, the pod 200 further comprises an indicator for identifying at last one of: the pod/capsule type; the nature and type of the herb-based filling, usage temperature profile, heater resistance at room temperature, production % expiration date, batch number, and tractability information. In specific embodiments, the envelope 202 is heat-isolated, optionally by using air cavities and optionally also sealers 203.

In specific embodiments the present invention provides a pod % capsule 200 comprising a capsule part comprising: (a) an envelope 202; and (b) a heater 201 designed for uniform heating of the herb-based filling and disposed within said envelope 202, the envelope 202 being filled with a wax filling with an herb-based active substance, suitable for inhaling said active substance using an electric inhaler 100, wherein the filling optionally comprises additives for heat conductivity, aerosol generation and/or anti-bacterial properties. In further specific embodiments, the pod 200 further comprises an indicator for identifying at last one of: the pod/capsule type; the nature and type of the herb-based filling, usage temperature profile, heater resistance at room temperature, production/expiration date, batch number, and tractability information. In specific embodiments, the envelope 202 is heat-isolated, optionally by using air cavities and optionally also sealers 203.

The pod 200 according to any one of the embodiments above is intended to be used by a dedicated inhaler that is designed to receive the pod 200 and upon activation thereof to provide the heater 201 with electricity. Accordingly, in a second aspect, the present invention provides an electric inhaler 100 for inhaling vapors of an herb-based active substance encapsulated/stored within a pod/capsule 200 as defined above holding a dried herb-based filling with said active substance, wherein said electric inhaler 100 comprises: (a) a hollow body having an airflow inlet and outlet 104 connected via an airflow path, wherein said airflow path is optionally replaceable; (b) a pod/capsule socket/receptacle 105 within said hollow body adapted to receive and hold said pod/capsule 200; (c) a mouthpiece located at the airflow outlet 104, said mouthpiece is optionally replaceable; and (d) an electrical power control switch, wherein the inhaler 100 is designed to receive and hold a pod/capsule 200 as defined above, and activate the heater 201 therewithin using an electric power source 101 to thereby release the active substance from the herb-based filling.

In specific embodiments, the inhaler 100 further comprises an internal power source, optionally rechargeable, in which case the inhaler 100 further comprises a charging connector/port, and optionally a charging-indicator.

In specific embodiments, the inhaler 100 further comprises a barcode or other type of memory reader for reading a barcode or other type of memory on the pod 200 for identifying at last one of: the pod/capsule type; the nature, type and amount of the herb-based filling, usage temperature profile, heater resistance at room temperature, production/expiration date, batch number, and tractability information.

The concept of using a heater resistance to measure the temperature is known, e.g., from JUUL™ or IQOS™ devices. However, using a heater as part of a disposable component raises several problems when trying to measure the temperature: (i) temperature variance—the temperature is read by measuring a percentage change from room temperature resistance and using the temperature coefficient of resistance of the material, calculating the temperature at any given time. Variations during the production can lead to a high variance in room temperature resistance (1% error in baseline resistance would lead to an error of around 10 degrees!). (ii) power consumption—since high power is required, low resistance and high current heaters are needed. This means that the contact resistance plays a major factor that cannot be read by the device. Accordingly, the present invention overcome all of these problems (and others) either by recording the baseline resistance of the pod during its production and storing it therein (i.e. in/on the consumable pod) in a way that can be read by the inhaler immediately prior to use; or by adding a sensor(s) to the inhaler, which enables compensation for these errors, e.g. adding 2 more contacts between the inhaler and the consumable pod to allow sensing of contact resistance during use, and adding a temperature sensor near the consumable pod to compensate for a starting temperature that is not room temperature, referred to herein as “heater resistance at room temperature”.

In certain embodiments, the electric inhaler 100 of the invention further comprises at least one of: an activation button, a security activation mechanism, LED indicator, a pod/capsule locking and release mechanisms, a temperature control element, and a pressure sensor (absolute or differential) that is designed to identify the onset and conclusion of an inhalation session to thereby enable the inhaler to know when to activate the heater and when to reduce the temperature of the heater 201 to preserve active substance and/or to allow placing the device aside when not in immediate use without the risk of creating a fire % burr.

In specific embodiments, the security activation mechanism is a fingerprint scanner, a DNA (from saliva) scanner, a facial recognition device, NFC (near field communication), dedicated application within a smartphone, or any other mechanism that enables identification of the user prior and/or during the activation of the inhaler 100.

In certain embodiments, the electric inhaler 100 of the invention further comprises a computer 102 comprising a processor and memory, wherein said memory is designed to record and store data about: user identification information; pod/capsule identification information, including the pod/capsule filling identification information, usage times and type, different operation methods (temperatures), pod/capsule types, usage temperature profile, heater resistances at room temperature, wherein the computer 102 is also designed to execute commands according to data received and stored, and has optionally a wireless communication means (e.g. via Bluetooth) for connecting thereof to a remote computer. For instance, once a pod 200 is placed within the pod socket 105, the, e.g., barcode reader provides the computer data that identifies the type of pod, the type of filling, and the desired usage program (e.g. in terms of temperature and duration), thereby enabling the computer to execute commands that enable proper activation of the heater 201 to achieve maximum and efficient usage of the herb-based filling in the pod 200. Another example is when the computer receives data from a user identification mechanism, compares it to stored data, and according to the results of the comparison execute commands that either enable activation of the inhaler or not.

In certain embodiments, the electric inhaler 100 of the invention is designed to measure the temperature within the pod/capsule 200 for enabling control thereof, e.g. via a temperature control element and/or the electrical power control switch.

Notably, in known devices and systems, the temperature within a pod is predefined and does not change during use. Accordingly, in certain embodiments, the electric inhaler 100 of the invention changes the temperature within the pod 200 during use as well as during the actual inhalation session for improving evaporation, overall experience of the user, prolong use of each pod, and reduce energy consumption.

In certain embodiments, the electric inhaler 100 of the invention further comprises a temperature sensor for measuring the inhaler's temperature and enabling turning the device off when the temperature is outside a predefined temperature range.

In certain embodiments, the present invention provides an electric inhaler 100 for inhaling vapors generated from an herb-based active substance encapsulated/stored within a pod/capsule 200 as defined above holding an herb-based filling with said active substance, wherein said electric inhaler 100 comprises: (a) a hollow body having an airflow inlet and outlet 104 connected via an airflow path, wherein said airflow path is optionally replaceable; (b) a pod/capsule socket/receptacle 105 within the hollow body adapted to receive and hold said pod/capsule 200; (c) a mouthpiece located at the airflow outlet 104, said mouthpiece is optionally replaceable; (d) an electrical power control switch; (e) an internal power source, optionally rechargeable; (f) a computer 102 comprising a processor and memory designed to record and store data and execute commands; (g) a barcode or other type of memory reader; (h) optionally, a temperature sensor for measuring the inhaler's temperature; and (i) at least one of: an activation button, a security activation mechanism, LED indicator, a pod/capsule locking and release mechanisms, a temperature control element, and a pressure sensor (absolute or differential), wherein the inhaler 100 is designed to receive and hold a pod/capsule 200 as defined above, and activate the heater 201 therewithin using an electric power source 101 to thereby release the active substance from the herb-based filling. In specific embodiments, the inhaler 100 is designed to measure the temperature within the pod/capsule 200 for enabling control thereof.

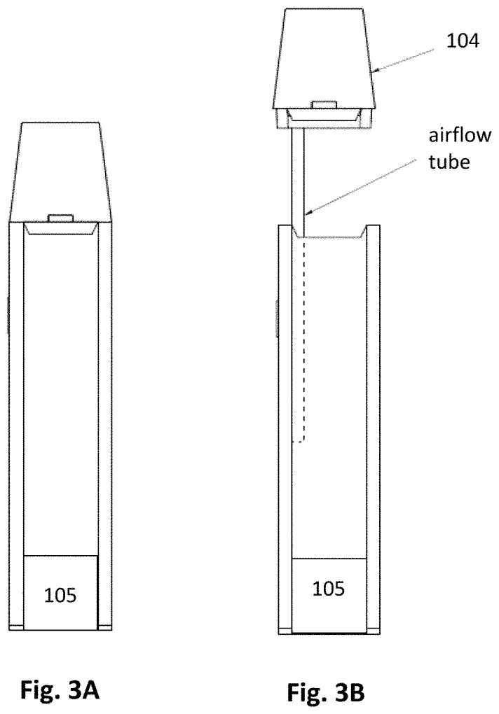

In certain embodiments of the electric inhaler 100 of the invention, the mouthpiece is replaceable, which improves hygiene and reduces the risk of accumulation of residues, clogging and contamination. In specific embodiments, the replaceable mouthpiece is connected to an airflow tube that is designed to deliver the vapor/fumes with the active agent(s) from the pod to the mouthpiece (see e.g. FIG. 3), i.e. the mouthpiece and the airflow path constitute a single unit separable from the hollow body. The mouthpiece and the airflow path are designed to fit the airflow inlet and outlet 104 of the inhaler 100. In such a configuration, when the mouthpiece is replaced, also the airflow tube is replaced, which further reduces the risk of contamination and clogging of the vapor/fumes passage. A replaceable mouthpiece enables using a narrower airflow channel without the risk of it being clogged, which further assists in cooling the inhaled aerosol.

In a third aspect, the present invention provides an electric inhaling system for consuming an herb-based active substance, the system comprises: (a) a pod/capsule 200 according to any one of the embodiments above; (b) an electric inhaler 100 according to any one of the embodiments above; and (c) a power source 101 (optionally rechargeable), optionally integrated within the electric inhaler 100.

In specific embodiments, the electric inhaling system of the invention for consuming an herb-based active substance comprises: (a) a pod/capsule 200 comprising a capsule part filled with a solid herb-based filling with an herb-based active substance, suitable for inhaling said active substance using an electric inhaler 100. In specific embodiments, the capsule part of the pod 200 comprises (a) an envelope 202; and (b) a heater 201 disposed within said envelope 202; (b) an electric inhaler 100 for inhaling vapors an herb-based active substance encapsulated/stored within a pod/capsule 200 as defined above holding a solid herb-based filling with said active substance, wherein said electric inhaler 100 comprises: (a) a hollow body having an airflow inlet and outlet 104 connected via an airflow path; (b) a pod/capsule socket/receptacle 105 within said body adapted to receive and hold said pod/capsule 200; (c) a mouthpiece located at the airflow outlet 104; and (d) an electrical power control switch, wherein the inhaler 100 is designed to receive and hold a pod/capsule 200 as defined above, and activate the heater 201 therewithin using an electric power source 101 to thereby release the active substance from the herb-based filling; and (c) a power source 101 (optionally rechargeable), optionally integrated within the electric inhaler 100.

In specific embodiments of the electric inhaling system of the invention, the pod 200 comprises a heater 201 designed to enable efficient heat conduction to all areas of the filling, e.g. by shaping it in a unique and efficient manner, such as cylindrical-shaped; spring-like-shaped; serpentine-shaped; rod- or multiple rods-shaped; or cubical-shaped.

In a forth aspect, the present invention provides a method for consuming an herb-based active substance, the method comprising the steps of: (i) placing a pod/capsule 200 as defined in any one of the embodiments above within a pod socket/receptacle 105 of an inhaler 100 as defined in any one of the embodiments above; (ii) activating the heater 201 within the pod 200 to thereby release said herb-based active substance from the herb-based filling; and (iii) inhaling the released active substance by inhaling via the mouthpiece of the inhaler 100.

In specific embodiments, the present invention provides a method for consuming an herb-based active substance, the method comprising the steps of: (i) placing a pod 200 comprising a capsule part filled with a dried herb-based filling with an herb-based active substance, suitable for inhaling said active substance using an electric inhaler 100. In specific embodiments, the capsule part of the pod 200 comprises (a) an envelope 202; and (b) a heater 201 disposed within said envelope 202, within a pod socket/receptacle 105 in an electric inhaler 100 for inhaling vapor from a heated/burned herb-based active substance encapsulated/stored within a pod/capsule 200 as defined above holding an herb-based filling with said active substance, wherein said electric inhaler 100 comprises: (a) a hollow body having an airflow inlet and outlet 104 connected via an airflow path (b) a pod/capsule socket/receptacle 105 within said body adapted to receive and hold said pod/capsule 200; (c) a mouthpiece located at the airflow outlet 104; and (d) an electrical power control switch, wherein the inhaler 100 is designed to receive and hold a pod/capsule 200 as defined above, and activate the heater 201 therewithin using an electric power source 101 to thereby release the active substance from the herb-based filling; (ii) activating the heater 201 within the pod 200 to thereby release said herb-based active substance from the herb-based filling; and (iii) inhaling the released active substance by inhaling via the mouthpiece of the inhaler 100.

The method of the invention for consuming an herb-based active substance may be for medical purposes, or for a non-therapeutic purpose such as for recreational use or for cosmetic use. Accordingly, in certain embodiments, the method of the invention is a non-therapeutic method for consuming an herb-based active substance. In alternative embodiments, the method of the invention is a therapeutic method for consuming an herb-based active substance.

In specific embodiments of the above methods, the inhaler 100 comprises a computer 102 comprising a processor and memory, wherein step (ii) of activating the heater 201 is preceded by steps of: (Ia) identifying: the type of pod/capsule 200 placed within the inhaler 100 (also including whether it is used or not); the type of filling within said pod/capsule 200; and (Ib) determining the operation method of said heater 201 in terms of temperature and duration.

In certain embodiments, the above methods further comprise a step of identifying the user before allowing activation of the heater 201 and/or during its activation, said step is based on receiving user data and comparing same to previously stored user identification information within the memory of the computer 102.

In certain embodiments of the above methods, the heater 201 is heated to one temperature for releasing the herb-based active substance from the herb-based filling, and to a different temperature during inhalation step (iii). Notably, when inhaling it is desired that the heater 201 is activated at the desired temperature in order to efficiently release the active substance(s) from the herb-based filling. Accordingly, when inhaling, the heater is to be activated to reach a desired temperature. However, when not inhaling, there is no need to release active substance(s) from the filling, and thus the temperature is reduced to both preserve filling/material and to allow placing the inhaler 100 aside without the risk of unintentional burn/fire. In addition, the reduced temperature when not-inhaling allows a fast response to inhalation, i.e. bringing the temperature back to the desired one for inhaling.

In certain embodiments of the above methods, the temperature of the heater 201 is determined according to the type of the filling and the herb-based active substance.

In certain embodiments, the temperature of the heater 201 is controlled to be below the ignition temperature of the herb-based filling. In alternative embodiments, the temperature of the heater is controlled to be above the ignition temperature of the herb-based filling.

In certain embodiments, the temperature of the heater 201 is controlled to allow combustion of the herb-based filling. In alternative embodiments, the temperature of the heater is controlled to prevent combustion of the herb-based filling.

The below Table 1 provides examples of various herbs and preferred temperature for complete evaporation thereof:

TABLE 1

| Herb | Temperature (° C.) |
|---|---|
| Lavender | 130 |
| Chamomile | 190 |
| Green tea leaves | 180 |

It should be noted that the herb-based filling is not conveyed/moved across the heater 201, but rather the heater 201 generates heat that reaches all the filling within the pod 200 without conveying the filling, i.e. the heat reaches all areas within the pod 200 that hold the filling.

In the following detailed description of various embodiments, reference is made to the accompanying drawings that form a part thereof, and in which are shown by way of illustration specific embodiments in which the invention may be practiced. It is understood that other embodiments may be utilized and structural changes may be made without departing from the scope of the present invention.

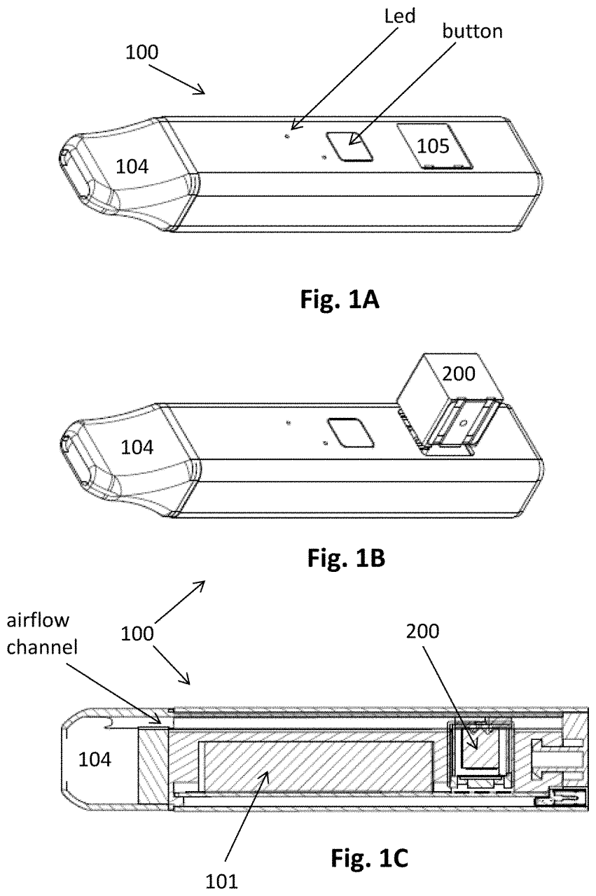

FIGS. 1A-1C illustrate one configuration of an inhaler 100 according to the invention is an elongated tube, optionally square-shaped with a narrow mouthpiece 104 adapted for placement in the mouth for easy inhalation. At the end of the inhaler that is remote from the mouthpiece there is a socket 105 adapted to receive a pod 200. The size and shape of the socket 105 are adapted to receive the pod 200. In certain embodiments, the socket 105 and/or the pod 200 comprise at least one sealing gasket (not shown) to prevent passage of air from the sides of the pod 200 when placed in the socket 105, thereby enabling passage of air only from within the pod 200 via the herb-based filling to enable withdrawal of fumes/vapors of the active agent(s) that is released from the filling after heating thereof. As illustrated, when inhaling, air enters the inhaler 100 via the pod 200 placed within its socket 105, and passes through an air-passageway (airway) within the inhaler 100 to and out from the mouthpiece 104.

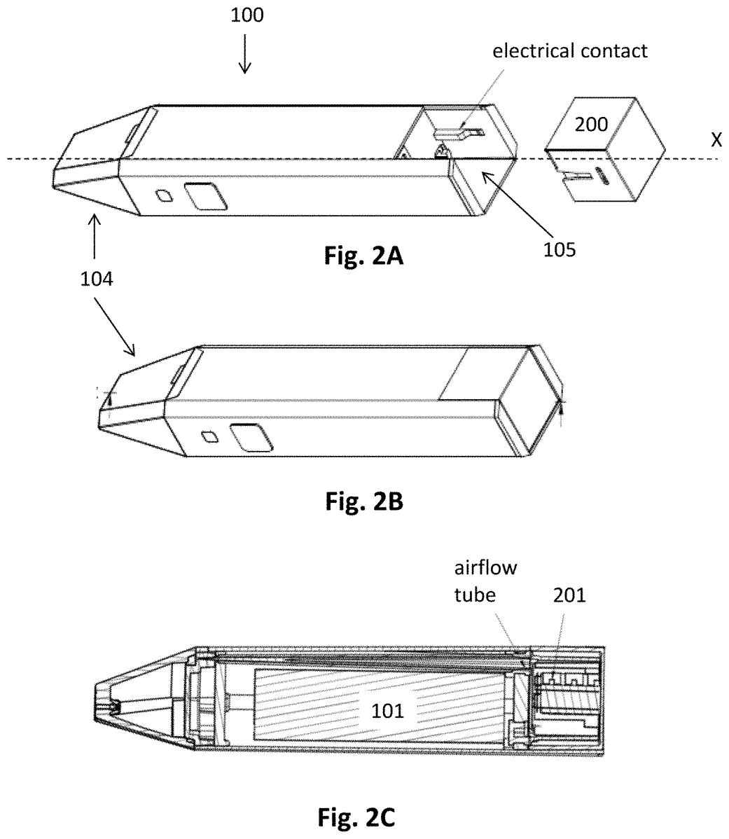

FIGS. 2A-2C illustrate another configuration of an inhaler 100 according to the invention, in which the socket 105 is at the opposite side of the inhaler relative to the mouthpiece 104, and the insertion and removal of a pod 200 is carried out along axis X.

FIGS. 3A-3B illustrate yet another configuration of an inhaler 100 according to the invention, in which the mouthpiece is replaceable and connected to an airflow tube connecting the pod 200 to the mouthpiece.

In certain embodiments, the same looking capsule 200 can represent different capsules 200, i.e. filled with a different type of herb-based filling for different use thereof. In such embodiments, each pod 200 comprises, e.g., a barcode that identifies the pod's type to the inhaler 100 thereby enabling to activate the inhaler according to the content of the pod, e.g., a dried herb filling designed for full combustion (meaning high temperature suitable for combustion, i.e. about 230° C.); a dried herb filling designed for evaporation (meaning that the heater will reach a temperature of about 180-190° C.); or wax filling designed for evaporation.

FIG. 4A illustrates a cross-sectional side-view of an alternative exemplary cubical pod 200, showing a heater 201 (wrapped around a dried-herb compact filling). FIG. 4B illustrates a bottom view of the pod 200, illustrating air cavities within the envelope/walls of the pod, designed to provide heat-insolation, and also an airflow passage around the dried-herb compact filling residing inside the pod.

In certain embodiments, the cubical pod 200 of the invention comprises a coiled heater 201 (wrapped around a dried-herb compact filling). In certain embodiments, the pod comprises an air passageway within the dried-herb compact filling residing inside the pod. In certain embodiments, the pod comprises air cavities within the envelope/walls 202 of the pod, designed to provide heat-insolation.

FIG. 5A illustrates an exploded view of a pod 200 according to one embodiment of the invention, showing an envelope with air-pockets for heat insolation; two sealers/gaskets 203 for keeping the air within the air-pockets; a herb-based dried-herb compact filling within a heater 201; external electric contacts/conduits (in this figure they are positioned on opposite sides of the pod, but it should be understood that they can be located on the same wall of the pod) that are connected to the heater 201, wherein the electric conduits and sealers 203 have an air passage hole for allowing air to enter the filling during inhalation. FIGS. 5B and 5C illustrate an exploded view of a pod 200 according to another embodiment of the invention, showing an envelope (top cover) and an insolation layer for heat insolation: a herb-based dried-herb compact filling; a heater 201; external electric conduits (in this figure they are positioned on the same side of the pod) that are connected to the heater 201.

FIGS. 7A-7E illustrate another configuration of the pod 200 of the invention where the heater and contacts are made out of stamped sheet metal. FIG. 7A shows sub-assembly of a base heater and filling. FIG. 7B shows an assembled pod. FIG. 7C is a cross section of the pod illustrating the air cavities trapped between the pod's parts/components. FIG. 7d illustrates how the solid herb filling is located inside the heater. FIG. 7E illustrates the airflow passage through the pod, i.e. entering from one side of the filling/heater assembly and exiting from the other end.

Many alterations and modifications may be made by those having ordinary skill in the art without departing from the spirit and scope of the invention. Therefore, it must be understood that the illustrated embodiment has been set forth only for the purposes of example and that it should not be taken as limiting the invention as defined by the following invention and its various embodiments and/or by the following claims. For example, notwithstanding the fact that the elements of a claim are set forth below in a certain combination, it must be expressly understood that the invention includes other combinations of fewer, more or different elements, which are disclosed in above even when not initially claimed in such combinations. A teaching that two elements are combined in a claimed combination is further to be understood as also allowing for a claimed combination in which the two elements are not combined with each other, but may be used alone or combined in other combinations. The excision of any disclosed element of the invention is explicitly contemplated as within the scope of the invention.

The words used in this specification to describe the invention and its various embodiments are to be understood not only in the sense of their commonly defined meanings, but to include by special definition in this specification structure, material or acts beyond the scope of the commonly defined meanings. Thus if an element can be understood in the context of this specification as including more than one meaning, then its use in a claim must be understood as being generic to all possible meanings supported by the specification and by the word itself.

The definitions of the words or elements of the following claims are, therefore, defined in this specification to include not only the combination of elements which are literally set forth, but all equivalent structure, material or acts for performing substantially the same function in substantially the same way to obtain substantially the same result. In this sense it is therefore contemplated that an equivalent substitution of two or more elements may be made for any one of the elements in the claims below or that a single element may be substituted for two or more elements in a claim. Although elements may be described above as acting in certain combinations and even initially claimed as such, it is to be expressly understood that one or more elements from a claimed combination can in some cases be excised from the combination and that the claimed combination may be directed to a sub-combination or variation of a sub-combination.

Insubstantial changes from the claimed subject matter as viewed by a person with ordinary skill in the art, now known or later devised, are expressly contemplated as being equivalently within the scope of the claims. Therefore, obvious substitutions now or later known to one with ordinary skill in the art are defined to be within the scope of the defined elements.

The claims are thus to be understood to include what is specifically illustrated and described above, what is conceptually equivalent, what can be obviously substituted and also what essentially incorporates the essential idea of the invention.

Although the invention has been described in detail, nevertheless changes and modifications, which do not depart from the teachings of the present invention, will be evident to those skilled in the art. Such changes and modifications are deemed to come within the purview of the present invention and the appended claims.

1-38. (canceled)

39. A pod/capsule 200 suitable for use with an electric inhaler 100, said pod 200 comprising: an envelope 202 and a heater 201, wherein:
said envelope 202 comprises a thermally isolated cavity that contains a solid herb-based filling with an herb-based active substance;
said envelope 202 comprises an airflow inlet and an airflow outlet designed to direct and allow passage of air via said cavity; and
said heater 201 is disposed within said cavity, and is designed to heat the cavity and said solid herb-based filling.

40. The pod/capsule 200 of claim 39, wherein said solid herb-based filling is held essentially entirely by said heater 201.

41. The pod/capsule 200 according to claim 39, wherein said solid herb-based filling is not in direct contact with said heater 201.

42. The pod/capsule 200 according to claim 39, wherein said heater 201 is cubical-shaped, wherein said heater 201 is permeable to vapor flow, such that the airflow passes through a narrow region around/within the heater 201, optionally via said filling, and wherein said solid herb-based filling is positioned around and/or within said heater 201.

43. The pod/capsule 200 according to claim 39, further comprising an indicator for identifying heater baseline resistance at room temperature and at least one of: the pod/capsule type; the nature and type of the solid herb-based filling, usage temperature profile, production/expiration date, batch number, and tractability information.

44. The pod/capsule 200 according to claim 39, wherein said envelope 202 comprises air cavities acting as heat-insulates.

45. The pod/capsule 200 according to claim 44, further comprising sealers 203 designed to maintain air within said air cavities.

46. The pod/capsule 200 according to claim 39, further comprising sealers 203 designed to direct and allow passage of air via said envelope 202.

47. The pod/capsule 200 according to claim 39, wherein said heater 201 is made of a material enabling temperature measurement inside the pod/capsule 200.

48. The pod/capsule 200 according to claim 39, further comprising at least two additional electric contacts to allow sensing of contact resistance during use.

49. The pod/capsule 200 according to claim 39, which is made, at least partially, of: permeable material that allows passage of air and of vapors/fumes of the active substance; or a material that upon activation of the heater 201, becomes permeable thereby allowing passage of air and of vapors/fumes of the active substance.

50. The pod/capsule 200 according to claim 39, wherein said envelope 202 is heat-isolated to maintain the pod's outer surface below 60° C. while heating said cavity.

51. The pod/capsule 200 according to claim 39, wherein air that flows from said inlet to said outlet of the envelope 202 does not pass through said solid herb-based filling

52. The pod/capsule 200 according to claim 39, wherein said envelope 202 is heat insulated and comprises air cavities acting as heat-insulates and sealers 203 designed to maintain air within said air cavities, wherein tearing of said sealers 203 damages the cavities' heat-insulate capabilities.

53. The pod/capsule 200 according to claim 39, further comprising readable data regarding baseline resistance of the pod.

54. An electric inhaler 100 for inhaling vapors of an herb-based active substance encapsulated/stored within a pod/capsule 200 as defined in claim 39 holding a solid herb-based filling with said active substance, wherein said electric inhaler 100 comprises:
a) a hollow body having an airflow inlet and outlet 104 connected via an airflow path;
b) a pod/capsule socket/receptacle 105 within said hollow body adapted to receive and hold said pod/capsule 200;
c) a mouthpiece located at the airflow outlet 104;
d) an electrical power control switch; and
e) a computer 102 comprising a processor and memory, wherein said memory is configured to record and store data about pod/capsule identification information and types, different operation methods (temperatures), usage temperature profiles, and/or heater resistances at room temperature,
wherein the inhaler 100 is designed to receive and hold a pod/capsule 200 and activate the heater 201 therewithin using an electric power source 101 to thereby release the active substance from the solid herb-based filling.

55. The electric inhaler 100 according to claim 54, further comprising a barcode or other type of memory reader for reading a barcode or other type of memory from a pod 200 placed within the inhaler, for identifying at last one of: the pod/capsule type; the nature, type and amount of the herb-based filling, usage temperature profile, heater resistance at room temperature, production/expiration date, batch number, and tractability information.

56. The electric inhaler 100 according to claim 54, further comprising a security activation mechanism and at least one of: an activation button, LED indicator, a pressure sensor (absolute or differential), a pod/capsule locking and release mechanisms, and a temperature control element, wherein said security activation mechanism is a fingerprint scanner, a DNA (from saliva) scanner, a facial recognition device, near field communication (NFC), or dedicated application within a smartphone, etc.

57. The electric inhaler 100 according to claim 54, further comprising a temperature sensor and configured to measure the temperature within the pod/capsule 200 for enabling control thereof.

58. The electric inhaler 100 according to claim 54, wherein said mouthpiece and said airflow path constitute a single unit separable from said hollow body and designed to fit said airflow inlet and outlet 104, wherein said single unit is optionally disposable.

59. An electric system for consuming a solid herb-based active substance, said system comprising:
a) a pod/capsule 200 according to claim 39 comprising a capsule part filled with a solid herb-based filling with an herb-based active substance, suitable for inhaling said active substance using an electric inhaler 100;
b) an electric inhaler 100 for inhaling vapors of an herb-based active substance encapsulated/stored within said pod/capsule 200 holding an herb-based filling with said active substance, wherein said electric inhaler 100 comprises: a hollow body having an airflow inlet and outlet 104 connected via an airflow path; a pod/capsule socket/receptacle 105 within said body adapted to receive and hold said pod/capsule 200;
a mouthpiece located at the airflow outlet 104; and an electrical power control switch, wherein the inhaler 100 is designed to receive and hold said pod/capsule 200, and activate the heater 201 therewithin using an electric power source 101 to thereby release the active substance from the solid herb-based filling; and
c) a power source 101 (optionally rechargeable), optionally integrated within said electric inhaler 100.

60. A method for consuming an herb-based active substance, said method comprising the steps of:
a) placing a pod/capsule 200 according to claim 39 within a pod/capsule socket/receptacle 105 in an inhaler 100;
b) identifying by the computer 102 the type of pod/capsule 200 placed within the inhaler 100 and the type of filling within said pod/capsule 200;
c) determining the operation method of said heater 201 in terms of temperature and duration according to the type of the filling and the solid herb-based active substance;
d) activating the heater 201 within said pod/capsule 200 to thereby release said herb-based active substance from the solid herb-based filling; and
e) inhaling the released active substance by inhaling via the mouthpiece of the inhaler 100.

61. The method of claim 60, further comprising a step of identifying the user before allowing activation of the heater 201, said step is based on receiving user data and comparing same to user identification information pre-stored within the memory of the computer 102.

62. The method of claim 60, wherein the heater 201 is heated to one temperature for releasing said herb-based active substance from the solid herb-based filling, and to a different temperature during inhalation step (e).
